(19) United States
(12) Patent Application Publication
(10) Pub. No.: US 2009/0258833 A1
(43) Pub. Date: Oct. 15, 2009

(54) FORTIFIED LIQUID PROTEIN COMPOSITIONS AND METHODS OF MAKING SAME

(76) Inventor: Edward Walker, Ogden, UT (US)

Correspondence Address: Workman Nydegger;1000 Eagle Gate Tower, 60 East South Temple, Salt Lake City, UT, 84111, US

(21) Appl. No.: 12/422,188

(22) Filed: Apr. 10, 2009

Related U.S. Application Data
(60) Provisional application No. 61/043,937, filed on Apr. 10, 2008.

(51) Int. Cl. A61K 8/65

(52) U.S. Cl. 514/12

(57) ABSTRACT

A fortified liquid protein composition and method includes hydrolyzed collagen and a desired abundance of sulfur containing amino acids and optionally taurine.

### CROSS-REFERENCE TO RELATED APPLICATIONS

This Application claims the benefit of U.S. Provisional Patent Application Ser. No. 61/043,937, entitled “Fortified Liquid Protein Compositions,” filed Apr. 10, 2008, which is hereby incorporated herein by reference.

### BACKGROUND OF THE INVENTION

1. The Field of the Invention

The disclosed embodiments relate to liquid protein compositions that are fortified with sulfur-containing amino acids.

2. Related Technology

Protein-energy malnutrition and inflammation, together also known as the malnutrition-inflammation complex syndrome (MICS), have been implicated as the most powerful death indicators in maintenance haemodialysis (MHD) patients. (Kalantar-Zadeh K, Block G, Humphreys M H, Kopple J D. “Reverse epidemiology of cardiovascular risk factors in maintenance dialysis patients.” Kidney Int 2003; 63: 793-808). Hypoalbuminaemia is a marker of MICS and a strong predictor of cardiovascular (CV) death in MHD patients. Many epidemiological studies have shown a strong association between serum albumin and prospective mortality, including CV death, in MHD patients (Kaysen G A, Dubin J A, Muller H G, Mitch W E, Rosales L M, Levin N W. “Relationships among inflammation, nutrition and physiologic mechanisms establishing albumin levels in hemodialysis patients,” Kidney Int 2002; 61: 2240-2249). Progressively increasing serum albumin values over time have been associated with improved outcomes. (Kalantar-Zadeh, et al., “Revisiting mortality predictability of serum albumin in the dialysis population: time dependency, longitudinal changes and population-attributable fraction,” Nephrology Dialysis Transplantation, Vol. 20, Number 9, pp. 1880-1888). Furthermore, that study indicated the survival advantages of serum albumin >3.8 g/dl and its incremental association with decreasing CV death up to a serum albumin of ≧4.4 g/dl. The study further suggests that it may be time for clinical trials of albumin-increasing interventions in MHD patients including nutritional supplements with or without anti-inflammatory or anti-oxidant properties.

### BRIEF SUMMARY OF THE INVENTION

The present invention relates to a fortified liquid protein composition that includes hydrolyzed collagen and a desired abundance of sulfur containing amino acids and optionally taurine. The compositions have been shown to provide substantial health benefits to healthy individuals and individuals with a protein deficiency. The fortified liquid protein compositions are readily digestible, which makes them suitable for those who are in need of a quick source of protein and/or individuals with maladies that limit the individual's ability to digest food.

The fortified liquid protein compositions include at least hydrolyzed collagen and a supplemental protein that includes sulfur containing amino acids. The fortified protein compositions are provided as a liquid solution. Liquid protein solutions are much easier for people to consume than powdered formulations. This is especially true for consumers who have difficulty in chewing or swallowing dry foods. For example, patients recovering from surgery or physiological trauma may have difficulty in eating normal foods. Liquid protein is an easy way to assimilate protein.

To enhance solubility of the collagen protein, typically the collagen is hydrolyzed into smaller polypeptides and individual amino acids. This technique not only enhances solubility of the collagen protein, but it also acts as a “pre-digestion” treatment that helps to facilitate absorption of amino acids in consumers who suffer from compromised digestive processes.

Collagen protein is an excellent source of the amino acids commonly found at higher concentrations in connective tissues such as skin and hair. Collagen has a unique tertiary structure that contributes to its exceptional tensile strength. Sufficient collagen is important to maintenance of healthy connective tissues. Collagen contains naturally high amounts of proline and hydroxyproline, as well as glycine and other amino acids that facilitate formation of its distinctive tissue structures. While amino acids such as glycine and proline are generally useful sources of protein, their relatively high concentration in collagen reduces the relative concentration of other amino acids. For example, concentrations of two sulfur-containing amino acids, methionine and cysteine, are conspicuously low in collagen.

When collagen is used as the primary source of dietary protein, a supplemental protein mixture is usually added to offset the particular amino acid deficiencies found in collagen. However, in contrast the present invention, collagen based supplements known in the art often include a majority of a natural protein such as whey protein or soy protein and/or hydrolyzed whey or soy protein. However, whey and soy protein can be difficult to formulate in a liquid protein mix due to its lower solubility and tendency to increase viscosity. In addition, hydrolyzed whey protein is known to taste bad in high concentration. In some embodiments of the invention, obtaining a relatively good tasting protein supplement is important because the taste of the supplement affects whether a patient or individual will stick with a prescribed treatment and thereby obtain the benefits of the treatment. Protein supplements that are repulsive to patients and individuals tend to be less effective simply because compliance with the treatment or usage is low. Therefore, in one embodiment, the fortified liquid protein compositions described herein can be relatively high in collagen content as compared to hydrolyzed whey or soy. In addition, the fortified protein compositions can include sweeteners and flavorings.

In the present invention, it has been found that collagen-based supplement can be substantially improved by fortifying a hydrolyzed collagen-based supplement with extra sulfur-containing amino acids, including methionine, cysteine, and derivatives thereof. Optionally, taurine and tryptophan can also be included.

The novel collagen based supplements of the present invention provide a more complete nourishment compared to existing collagen based supplements while retaining the beneficial properties of known collagen based supplements. These advantages are achieved by forming a liquid solution that includes hydrolyzed collagen and at least about 1.0 wt % (based on total protein content) of sulfur containing amino acids, such as methionine and cysteine.

In one formulation, the sulfur-containing amino acids, such as methionine and cysteine, are added in amounts so that their concentrations are two to ten times higher than normally present when only collagen is dissolved at identical concentrations. As a result, consumers now have an enhanced supply of methionine and cysteine to support their nutritional regimen.

Studies performed with fortified protein compositions according to one embodiment described herein have been shown to be highly effective at improving the health of malnourished individuals. Surprisingly, individuals consuming fortified liquid protein compositions with relatively high sulfur containing amino acids have been shown to have increased levels of albumin, which is an indication of the salubrious effects of the fortified liquid protein compositions.

These and other objects and features of the present invention will become more fully apparent from the following description and appended claims, or may be learned by the practice of the invention as set forth hereinafter.

### DETAILED DESCRIPTION

For purposes of this invention, the terms “sulfur-containing amino acid” and “derivatives of sulfur containing amino acids” do not include “taurine.”

For purposes of this invention, calculations involving the weight of hydrolyzed collagen do not include bonded water (i.e., the weight of the hydrolyzed collagen is calculated based on the weight of the amino acid content).

The fortified protein compositions described herein are liquid compositions that include a significant amount of protein. Typically the liquid solution includes at least about 25% by weight of protein (i.e., total weight of protein to total weight of liquid composition). Alternatively the protein content of the liquid solution can be at least about 35% or at least about 45%. In one embodiment, the protein content is in a range from about 25% to about 65%, alternatively in a range from about 35% to about 60%, or in a range from about 45% to about 55% of the total composition weight.

The liquid solution of the fortified liquid protein composition can include all or a portion of the following components: (i) hydrolyzed collagen, (ii) supplemental protein mixture (e.g., sulfur containing amino acids), (iii) taurine, (iv) tryptophan, (v) sweetener, and/or (vi) flavoring.

The collagen useful in the fortified liquid protein compositions is provided in a liquid form or is hydrolyzed to form a liquid. Any source of collagen can be used so long as the collagen can be liquefied at a desired concentration. Collagen is typically provided from the body tissues of porcine or bovine carcasses. Collagen is the main protein of connective tissue in animals and the most abundant protein in mammals, making up about 25% to 35% of the whole-body protein content. While porcine and bovine collagen are typically used, the invention is not limited to these particular sources of collagen.

The collagen can be liquefied through hydrolysis. For purposes of this invention, the term “hydrolysis” shall include “partial hydrolysis” and does not require “complete hydrolysis” unless otherwise specified. Hydrolysis of collagen can be carried out using an acid solution and/or proteolytic enzymes. The extent of hydrolysis typically depends on the acid concentration and duration of the hydrolysis, with lower pH and longer digestion periods resulting in more complete hydrolysis. Those skilled in the art are familiar with techniques for manufacturing hydrolyzed collagen. In one embodiment less than about 50 wt % of the collagen remains unhydrolyzed, alternatively less than about 30% or less than about 20% of the hydrolyzed collagen remains unhydrolyzed.

The hydrolyzed collagen is a major percentage of the overall weight percent of protein. In one embodiment, the weight percent of collagen of the total protein content of the liquid solution is at least about 40%, alternatively at least about 60%, 80%, 95%, or even 98%.

A supplemental protein mixture is included in the solution to fortify the collagen fraction. The supplemental protein mixture can include any type and/or amount of protein so long as it can be soluble in the liquid protein mix and includes one or more sulfur containing amino acids. Example of suitable sulfur containing amino acids includes cysteine, methionine, and derivatives of these. The sulfur containing amino acids, are typically provided as a soluble powder that can be mixed into the liquid solution. The sulfur containing amino acids can be added to the protein mix in a concentration of at least about 1.0 wt % (based on the total protein content), alternatively at least about 2.0 wt % or at least about 3.0 wt %.

These high concentrations of sulfur containing amino acids are not naturally occurring ratios of sulfur containing amino acids. By using a supplementary protein mixture that includes high concentrations of sulfur containing amino acids to fortify the collagen, a mixture with a non-natural or significantly increased concentration of sulfur containing amino acids can be achieved. As discussed below, this increased concentration of sulfur containing amino acids can have substantial health benefits, including, but not limited to increased albumin levels.

In one embodiment, the sulfur containing amino acid includes at least cysteine. Cysteine can be added in a concentration of at least 0.15 wt % (based on total protein content), or alternatively at least about 0.2 wt %, at least about 0.5 wt %, or even at least about 1.0 wt %.

In yet another embodiment, the sulfur containing amino acid includes at least methionine. The methionine can be added in an amount of at least about 0.75 wt % (based on total amino acid content), alternatively at least about 1.0 wt %, or at least about 2.0 wt %.

The liquid protein compositions can also include taurine. Taurine is an amine that has a sulfonic acid group. While the amine group of taurine can form a peptide bond, the sulfonic acid group cannot form a peptide bond and therefore taurine cannot extend the chain length of a polypeptide. Nevertheless, taurine is present at relatively high concentrations in cells and inter-cellular spaces in many tissues. Taurine can be made in vivo from sulfur containing amino acids such as cysteine and methionine. As such, in vivo synthesis of taurine can diminish the concentration of sulfur containing amino acids. It can be important for individuals to have an ample supply of cysteine and methionine to support taurine synthesis. Taurine can optionally be added to the liquid protein composition to minimize the conversion of cysteine and methionine to taurine in individuals consuming the liquid protein composition.

Since taurine is not present in collagen, any added taurine, in combination with sulfur-containing, naturally-occurring amino acids, methionine and cysteine, adds value to collagen-based protein products. In one embodiment, the fortified protein compositions can include taurine in a concentration range of 0.5-10% of the total protein present (0.5-10 g /100 g of total protein). In an alternative embodiment, taurine can be present in a range of 1.0-5% by weight of total protein.

Another optional amino acid that can be added to the liquid protein compositions is tryptophan. Tryptophan is an essential amino acid because individuals do not have the ability to synthesize tryptophan. Tryptophan can optionally be added to the fortified liquid protein composition in amounts from about 0.5-10 wt % of the total protein content.

The liquid protein compositions can also include other components that make the composition desirable to consume. For example, the liquid protein compositions can include sweeteners, flavoring, preservatives, colorings, etc. The sweeteners can be artificial, low calorie sweeteners or natural sweeteners such as granulated sugar or high fructose corn syrup. The sweeteners, flavorings, preservatives and/or food colorings can be added to the liquid solution in amounts suitable for providing desired taste for consumption by people.

The collagen, supplemental protein mix, sweetener, flavorings, preservatives, and food colorings are typically mixed together in water to form the liquid protein composition. Water can be included in amounts from about 25% to about 75% by weight of the total composition. Mixing can be carried out in a blender and/or using any mixing apparatus suitable for mixing collagen and amino acids. Because the composition is liquid and the protein is hydrolyzed, individuals who have difficulty eating solid foods can more easily consume the nutritional supplement as compared to traditional sources of protein such as meat.

In one embodiment, the fortified liquid protein composition can be used to treat a person that is malnourished, undernourished, or in need of additional protein nourishment (e.g., body builders that have a metabolically high demand for amino acids). In one embodiment, individuals in need of protein nourishment can be instructed to consume an effective amount of the fortified liquid protein at least once or twice a day for several weeks. Daily use of the composition over periods of several weeks have shown improved benefits in blood albumin levels.

Optimally, a convalescing patient who is recovering from trauma to the gastrointestinal tract can consume a relatively small quantity of liquid protein nutritional product of the invention. For example, one fluid ounce (approximately thirty milliliters) is a quantity that can be consumed without overtaxing the individual. The high concentration of protein allows the patient to take small amounts while still receiving substantial nourishment. In one embodiment the volume of a dose of the composition is in a range from about 0.5 fluid ounce to about 30 ounces, alternatively in range from about 0.5 ounces to about 12 ounces or about 1 ounce to about 8 ounces.

One fluid ounce is often an optimal volume of product for an elderly person who may exhibit only a slight appetite. In the invention, the formulation described contains significant quantities of protein and additional sulfur-containing amino acids to enhance its nutritional value. One fluid ounce of liquid protein composition can include 10-20 grams of hydrolyzed collagen protein. In addition, the same volume of the composition can include 0.15-0.75 g methionine, 0.0015-0.075 g cysteine, and 0.008-1.5 g taurine per fluid ounce of liquid protein formulation. These quantities of sulfur-containing amino acids represent enhanced quantities of the amino acids, methionine and cysteine, at levels of at least two to three of their typical concentration in collagen protein alone. In the case of taurine, this quantity is considerably more than 1,000 times the amount of taurine in unfortified aqueous solutions of this protein, since there is typically no taurine in natural collagen.

This novel addition of sulfur-containing amino acids to liquefied collagen protein represents a very desirable composition for nutritional supplementation of proteins, especially among nutritionally-compromised patients who are in need of fortified protein. This addition provides a new type of formulation that provides sulfur-containing amino acids normally missing in current nutritional products on the market today. The invention will be of especially great value to gastro-intestinally challenged patients who have unique protein supplementation needs.

### EXAMPLE

To test the effectiveness of the fortified liquid protein compositions, a clinical study was performed in conjunction with a continuous quality improvement (“CQI”) project. The study was carried out with patients receiving liquid protein after each treatment. The primary goal was to detect improvements in overall albumin levels. Secondarily, the study identified the participant acceptance of and tolerance to the particular liquid protein used.

The study was carried out using Provide® Sugar Free liquid protein supplement. The hydrolyzed collagen was derived from bovine protein. The protein content of the collagen content and the supplemental amino acids are shown below in Table 1.

TABLE 1

| | | AAs/ | | |
|---|---|---|---|---|
| | g/100 g | 1000 g | Extra Amino | Sum All |
| | Protein | Protein in | Acids Added | protein |
| | (including | Liq | g/1000 g Liq | g/1000 g Liq |
| Amino Acid | H₂O) | Prod: (g) | Prod | Prod |
| Alanine | 11.3 | 43.5 | | 43.5 |
| Arginine | 9 | 34.6 | | 34.6 |
| Aspartic Acid | 6.7 | 25.8 | | 25.8 |
| Cysteine | 0.08 | 0.3 | 0.65 | 1.0 |
| Glutamic Acid | 11.6 | 44.6 | | 44.6 |
| Glutamine | 0 | 0.0 | 2.13 | 2.1 |
| Glycine | 27.2 | 104.6 | | 104.6 |
| Histidine | 0.7 | 2.7 | 2.99 | 5.7 |
| Hydroxylysine | 0.76 | 2.9 | | 2.9 |
| Hydroxyproline | 13.3 | 51.1 | | 51.1 |
| Isoleucine | 1.54 | 5.9 | | 5.9 |
| Leucine | 3.45 | 13.3 | | 13.3 |
| Lycine | 4.36 | 16.8 | | 16.8 |
| Methionine | 0.63 | 2.4 | 2.56 | 5.0 |
| Phenylalanine | 2.49 | 9.6 | | 9.6 |
| Proline | 15.5 | 59.6 | | 59.6 |
| Serine | 3.73 | 14.3 | | 14.3 |
| Taurine | 0 | 0.0 | 14.23 | 14.2 |
| Threonine | 3.36 | 12.9 | | 12.9 |
| Tryptophan | 0 | 0.0 | 3.37 | 3.4 |
| Tyrosine | 0.29 | 1.1 | | 1.1 |
| Valine | 2.77 | 10.7 | | 10.7 |
| Totals: | 118.76 | 456.7 | 25.9 | 482.6 |

The first column of table 1 provides a list of amino acids with the second column indicating the relative abundance of the amino acids in a typical bovine collagen. The third column shows the weight of amino acids per gram of total liquid protein composition for the hydrolyzed collagen fraction. The fourth column indicates the amount of supplemental amino acids added to the liquid product according to one embodiment. The fifth column provides the total grams of protein per 1000 g of liquid product. The foregoing composition is referred to hereinafter as Sugar Free Provide.

Initially, thirty-four patients were identified to participate in the study. Patients with unstable albumin (defined as fluctuating from 4.0 g/dl or less, or consistently less than 4.0 g/dl) were eligible to participate. Of these, patients who did not consistently attend their hemodialysis treatments were eliminated. Nursing home patients were not included in the project. Twenty-eight patients participated to conclusion. Six patients dropped out at the beginning of the project. Four of the six objected to the taste of the particular composition used. Of the 28 participants, 82% (23 participants) had been on dialysis <90 days, with 17% of those having been on dialysis less than one year. One participant had been a dialysis patient less than 90 days, one was at 60 days, one at 30 days, and two less than 30 days. Nineteen women and nine men made up the 28 participants. Patients ranged in age from 47 years to 79 years. Eighteen of the participants had diabetes. One participant had diagnosis of malnutrition and failure to thrive. Still one other participant was bedridden and had numerous ubitus ulcers that were discovered around the time of the beginning of this project. Other than the end stage renal disease that required the patents to be on dialysis, none of the patients had any other serious or chronic illnesses.

Patients had previously been instructed to consume a high protein diet, in accordance with each patient's nutritional needs and the renal diet. For this project, patients were instructed to continue their current diet and nutritional supplement regimen at home. Each participant received 30 mL of Sugar Free Provide liquid protein after each treatment, immediately after their time on the dialysis machine had ended.

The time frame of the project encompassed a total of twenty-four treatments. Twenty-one per cent, or 6 patients, received all 24 doses of the Provide® Sugar Free liquid protein. Two patients refused on two occasions, d/t c/o nausea and/or upset stomach. Neither related their complaints to the liquid protein. One pt refused one time d/t diarrhea and upset stomach but stated it was not related to the product. Three patients were hospitalized during the term of the project. One started late d/t hospitalization and the other two were hospitalized after the project started. One of those two, the malnourished/failure-to-thrive patient, was hospitalized twice. These hospitalizations caused these patients to miss several doses. Two patients received less than 24 doses because one started dialysis after the project had commenced and began participation five treatment days into the project. The other patient withdrew from the project after the first day because she was already taking a lot of medicines and did not want to take anything else, including the nutritional supplement used in this project. She later rejoined the project, but had missed six servings in the meantime. The most doses missed by non-hospitalized patients, excluding the two just discussed, was four, by one patient only.

Increases in albumin were seen after the first month in 25% of the participants. These June labs showed improvements in albumin in seven patients. July labs showed that 43% of all the albumins improved. Of that 43%, one patient, or 8.3%, obtained an albumin of 4.0 g/dI, while 42% obtained albumins ≧3.8 g/dl but less than 4.0. During the entire term of the project, albumins improved by 68%.

After the first month of the project, eight participants reported poor intakes. One related to having an upset stomach for the three weeks prior, which caused her to be unable to eat. But, she said she was feeling better and was eating better at the time of lab review. She denied that her stomach discomfort was related to the liquid protein. Her albumin in the second month was 3.8. One participant reported poor appetite and inability to eat adequate portions. She had believed she was eating better but recall revealed wholly inadequate portions and 1-2 meals. Another patient reported inadequate intakes due to financial and living situation. Another patient had consistently poor intakes of HBV protein, eating junk food, and non-compliance with supplements. One reported failure to thrive. Two did not change their habits of eating protein, but ate in small amounts, which resulted in stable, but low albumin. The second month, one of these two increased his portions, which resulted in a 3.8 albumin. Three participates admitted that they were expecting the liquid protein to improve their albumin so they were eating less.

Most weights, 53.5%, remained stable during the project, while 28.6% of the participants experienced weight loss. In some cases, the loss was due to hospitalizations and, in the one mentioned case, limited accessibility to food.

Throughout the course of the project, no patient refused the Provide® Sugar Free liquid protein due to taste or tolerance. Ten out of twelve participants who expressed opinions on the Provide® Sugar Free v. a different type of liquid protein they had used in the past preferred the taste of the Provide® Sugar Free liquid protein.

After ending the project, the next month's labs revealed, of the improvements seen in the labs during the project, ten of those albumins dropped from where they had been when the project ended.

The results of this CQI project indicate that giving patients the liquid protein after each treatment may help to improve albumin outcomes through consistent intakes therefore improved compliance with nutritional supplements. Participants' tolerance and acceptance of the Sugar Free Provide liquid protein suggests that this nutritional supplement is an appropriate choice.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

We claim:

1. A liquid protein supplement, comprising:
a liquid solution including,
a hydrolyzed liquid collagen;
supplemental sulfur containing amino acids in an amount sufficient to give the liquid solution a weight percent of sulfur containing amino acids of at least about 0.75% (w/w), based on total protein content in the liquid solution; and
optionally taurine.

2. A liquid protein supplement as in claim 1, wherein the weight percent of sulfur containing amino acids is at least about 1.0% (w/w), based on the total protein content.

3. A liquid protein supplement as in claim 1, wherein the supplemental sulfur containing amino acids include methionine and/or a derivative of methionine and the weight percent of the methionine and/or the derivative of methionine is at least about 1.0% (w/w), based on the total protein content.

4. A liquid protein supplement as in claim 1, wherein the supplemental sulfur containing amino acids include cysteine and/or a derivative of cysteine and the weight percent of the cysteine and/or derivative of cysteine is at least about 0.15% (w/w), based on the total protein content.

5. A liquid protein supplement as in claim 1, wherein the hydrolyzed liquid collagen comprises at least about 40% (w/w) of the liquid solution.

6. A liquid protein supplement as in claim 1, wherein at least about 10 weight percent of the hydrolyzed liquid collagen is a polypeptide.

7. A liquid protein supplement as in claim 1, further comprising a sweetener and a flavoring.

8. A liquid protein supplement, comprising:
a liquid solution that includes,
at least about 25 wt % hydrolyzed liquid collagen, based on the total weight of the liquid solution;
at least about 0.15 wt % cysteine, based on the total protein content;
at least about 0.75 wt % methionine, based on the total protein content; and
at least about 1 w% taurine based on total protein content.

9. A liquid protein supplement as in claim 8, wherein the liquid solution includes at least about 35 wt % of the hydrolyzed liquid collagen, based on the total weight of the liquid solution.

10. A liquid protein supplement as in claim 8, wherein the liquid solution includes at least about 0.2 wt % cysteine, based on the total protein content.

11. A liquid protein supplement as in claim 8, wherein the liquid solution includes at least about 1.0 wt % methionine, based on the total protein content.

12. A liquid protein supplement as in claim 1, further comprising a sweetener and a flavoring.

13. A method for manufacturing a liquid protein supplement, comprising:
providing a hydrolyzed liquid collagen;
mixing methionine and/or cysteine with the hydrolyzed liquid collegen to obtain a liquid protein supplement enriched in sulfur containing amino acids, wherein the methionine and/or cysteine are added as an amino acid; and
optionally mixing taurine with the hydrolyzed liquid collagen.

14. A method as in claim 13, wherein the at least about 1.0 wt % methionine and/or cysteine in the form of an amino acid is mixed with the hydrolyzed liquid collagen, based on the total protein content.

15. A method as in claim 13, wherein the at least about 2.0 wt % methionine and/or cysteine in the form of an amino acid is mixed with the hydrolyzed liquid collagen, based on the total protein content.

16. A method as in claim 13, wherein the at least about 3.0 wt % methionine and/or cysteine in the form of an amino acid is mixed with the hydrolyzed liquid collagen, based on the total protein content.

17. A method as in claim 13, wherein the at least about 1.0 wt % taurine is mixed with the hydrolyzed liquid collagen, based on the total protein content.

18. A method as in claim 13, further comprising mixing a sweetener and a flavoring with the hydrolyzed liquid collagen.
